(19) United States
(12) Patent Application Publication
(10) Pub. No.: US 2004/0086135 A1
(43) Pub. Date: May 6, 2004

(54) Active noise control system using pure feedforward method with order-based offline calibration

(75) Inventor: Manish Vaishya, Auburn Hills, MI (US)

Correspondence Address: Elsa Keller, Siemens Corporation, Intellectual Property Department, 170 Wood Avenue South, Iselin, NJ, 08830, US

(73) Assignee: Siemens VDO Automotive Inc.

(21) Appl. No.: 10/672,529

(22) Filed: Sep. 26, 2003

Related U.S. Application Data
(60) Provisional application No. 60/423,088, filed on Nov. 1, 2002.

(51) Int. Cl. A61F 11/06; G10K 11/16; H03B 29/00

(52) U.S. Cl. 381/71.4; 381/86

(57) ABSTRACT

An active noise control system for controlling induction noise of an internal combustion engine having a speaker and a controller. The controller generates a control signal that drives the speaker, wherein the signal is based on at least one current vehicle operating condition. The signal is also based on a determination of a first sound pressure for each order of sound generated by the engine during a run up of the engine. In addition, the signal is based on a determination of a second sound pressure computed for each of a plurality of operating conditions of the engine, wherein the signal controls each of order of sound generated by the engine independently to drive the speaker to generate an audio output to control intake noise.

### CROSS REFERENCE TO RELATED APPLICATION AND PRIORITY CLAIM

This application claims the benefit of U.S. Provisional Application No. 60/423,088 (Attorney Docket No. 2002P18118US) filed on Nov. 1, 2002 in the name of Manish Vaishya and entitled FEEDFORWARD ORDER-BASED OPEN LOOP SYSTEM WITH PLANT COMPENSATION FOR ACTIVE NOISE CONTROL which is incorporated by reference herein in its entirety. This application is also related to nonprovisional application U.S. Serial No. (to be determined), Attorney Docket No. 2002P18118US01 filed on filed Sep. 25, 2003 in the name of Manish Vaishya and entitled INTAKE SOUND CONTROL USING PURE FEEDFORWARD METHOD WITH ORDER-BASED OFFLINE CALIBRATION which is hereby incorporated by reference herein in its entirety.

### FIELD OF THE INVENTION

This invention relates to noise control system for a vehicle, and more particularly, to a feedforward noise control system which generates a control signal based on sound pressures that are computed offline and which are order based.

### BACKGROUND OF THE INVENTION

Motor vehicle engines generate an induction sound which is primarily caused by the reciprocating motion of intake valves. Hence for a four stroke engine, a sound spectrum is generated that is comprised of the half order of the engine revolution (i.e. a full order of camshaft revolution) and higher harmonics thereof. Motor vehicles also include an air induction system having an intake orifice and a ducting arrangement which directs air from the environment to an internal combustion engine for use in a combustion process. A characteristic of such systems is that the induction sound travels through the induction system to the intake orifice in a finite amount of time which depends on duct length and other acoustic factors. The sound then propagates to the passenger compartment of the vehicle. This sound is undesirable for passengers inside the vehicle and contributes to environmental noise.

Frequently, the induction sound is attenuated by using passive devices such as Helmoltz and Quarter wave resonators, dampers and sound absorbers. A disadvantage with such devices is that they are only effective for relatively narrow frequency bands. In addition, they add non-uniformity to the resulting sound. Passive devices also tend to require relatively large amounts of space and are thus difficult to package within the tight confines of modern engine compartments. Further, such devices can only attenuate noise and cannot enhance sound.

Another technique for attenuating induction sound is through the use of an active noise cancellation system, such as a closed-loop active noise control system. Such systems utilize a Digital Signal Processor (DSP) to generate a control signal that is used to drive a speaker. The speaker then generates a sound adapted to attenuate undesirable noise so that it is reduced or cancelled. A microphone is used to monitor system performance. Information obtained from the microphone is then ultimately used to adjust various control parameters. Use of a DSP is necessitated in such systems due to the large number of computations that are needed.

Although fairly versatile, closed loop systems are expensive due to the auxiliary components that are required. In addition, the microphones utilized in such systems have many limitations and are not suited for use in an automotive environment. Closed loop systems also have limited tracking capability due to background noise and since they are based on a principle of gradual convergence. Further, such systems may become unstable under many situations due to feedback.

### SUMMARY OF THE INVENTION

An active noise control system for controlling induction noise of an internal combustion engine having a speaker and a controller is disclosed. The controller generates a control signal that drives the speaker, wherein the signal is based on at least one current vehicle operating condition. The signal is also based on a determination of a first sound pressure for each order of sound generated by the engine during a run up of the engine. In addition, the signal is based on a determination of a second sound pressure computed for each of a plurality of operating conditions of the engine, wherein the signal controls each of order of sound generated by the engine independently to drive the speaker to generate an audio output to control intake noise.

The features of the invention believed to be novel are set forth with particularity in the appended claims. The invention itself, however, both as to organization and method of operation, together with further objects and advantages thereof, may be best understood by reference to the following description taken in conjunction with the accompanying drawings.

### BRIEF DESCRIPTION OF THE DRAWINGS

FIG. 1 depicts a block diagram for a feedforward order based open loop control system in accordance with the present invention.

FIG. 2 illustrates an example of a set of look-up tables for temperature effects on control signal amplitude.

FIG. 3 shows a flow diagram depicting aspects of the present invention.

FIG. 4 is a schematic representation for a feedforward active noise control system.

### DETAILED DESCRIPTION OF THE INVENTION

While this invention is susceptible of embodiment in many different forms, there is shown in the drawings and will herein be described in detail specific embodiments, with the understanding that the present disclosure is to be considered as an example of the principles of the invention and not intended to limit the invention to the specific embodiments shown and described. In the description below, like reference numerals are used to describe the same, similar or corresponding parts in FIGS. 1-4.

Motor vehicle engines generate an induction sound which is primarily caused by the reciprocating motion of intake valves. Hence for a four stroke engine, a sound spectrum is generated that is comprised of the half order of the engine revolution (i.e. a full order of camshaft revolution) and higher harmonics thereof. In the present invention, each camshaft order is independently controlled so that any desired sound characteristic can be achieved.

Motor vehicles include an air induction system having an intake orifice and a ducting arrangement which directs air from the environment to an internal combustion engine for use in a combustion process. The induction sound travels through the induction system to the intake orifice in a finite amount of time which depends on duct length and other acoustic factors.

The engine intake sound at an orifice for any given order n is represented by:

\tilde{P}_{n,ω}=\tilde{P}_{n,ω}(h, T, Pₐ, L₁, k₁ . . . kₘ) Equation 1.

Here

\tilde{P}: complex sound pressure

n: order number

ω: camshaft rpm

h: throttle opening

T: mean air temperature

L₁: acoustic length from intake valves to orifice

Pₐ: ambient barometric pressure

kᵢ: miscellaneous engine control parameters

Referring to FIG. 1, a block diagram for a feedforward order based open loop control system 10 for active noise control is shown. In the present invention, a vehicle is run on a vehicle dynamometer while a microphone located at the intake orifice is used to measure the intake sound of the engine during a gradual run-up under a wide open throttle condition (block 12). The sound pressure is then computed for each order as a function of engine RPM (block 14). By way of example, the sound pressure for up to 32 frequency bands may be computed. This data is arranged as amplitude and phase with respect to a reference signal that is indicative of an angular position of a camshaft that drives the intake valves of the engine. In a preferred embodiment, the reference signal is obtained from a sensor that directly measures the angular position of the camshaft such as a sensor found in an engine management system utilized on many vehicles. In addition, a digital time capture may be used to measure a camshaft signal.

In the present invention, a speaker 22 is utilized which serves as a secondary source of sound for providing noise control. The speaker 22 may be a commercially available item whose size and characteristics are determined by the acoustic power requirements. In many instances, the speaker 22 will be sufficiently small so as to fit inside a vehicle air induction system. The speaker 22 is mounted at the intake orifice and forms part of a secondary path that includes associated electronic components and the same microphone that was used to measure the intake sound. It is noted that the microphone is used for calibration purposes and not for noise control. A calibration of the secondary path is performed offline by having the speaker 22 emit a broadband noise which is detected by the microphone. The frequency response at the microphone is then obtained.

The frequency response is given by:

\tilde{H}(f)=\tilde{H}(f, T, Pₐ, L₂) Equation 2.

Here

\tilde{H}: complex transfer function

f: signal frequency

L₂: acoustic length of secondary path

The baseline control signal is computed using the knowledge of the sound pressure (Equation 1) and the transfer function (Equation 2) as follows:

Ũ_{n,ω}=\tilde{H}⁻¹(f)×\tilde{P}_{n,ω} Equation 3.

Here Ũ: the baseline control signal.

The parameters n, ω and f are related by:

f=ω×n/60 Equation 4.

As all data is computed in frequency domain, the effect of \tilde{H}⁻¹(f) is equivalent to adding a phase and applying a gain to the amplitude (block 16). This helps in avoiding using extensive digital filters, which operate in typical time-domain systems. Thus, an [n₁×n₂] matrix table is obtained for each amplitude and phase, where n₁ is the desired number of controlled orders and n₂ is the number of rpm steps.

Once the baseline control signal is obtained, the system 10 is configured so that it is adaptable to a plurality of externally varying conditions that may occur such as temperature, ambient pressure, throttle opening, engine speed, and other vehicle specific engine control parameters that may affect engine sound. In order to achieve this, each condition is simulated and the resulting engine sound spectra is mapped and recorded. For instance, a recording is made for a part open throttle (for example, 75°), such that \tilde{P}′ₙ is the new sound spectrum at 75° throttle. This information is supplemented with the baseline values to obtain a correction factor as follows:

\tilde{C}_{75° throttle}=\tilde{P}′ₙ/\tilde{P}ₙ Equation 5 (block 18).

Since the system 10 is linear, the control signal is influenced by the same amount as the raw noise, so that:

$$\begin{matrix}{\tilde{U}}_{n , Ω}^{′} = {\tilde{U}}_{n , Ω} \times \tilde{C} \\ = \vert {\tilde{U}}_{n , Ω} \vert \times \vert {\tilde{P}}_{n , Ω}^{′} / {\tilde{P}}_{n , Ω} \vert \times [ ∠ {\tilde{U}}_{n , Ω} + ∠ ( {\tilde{P}}_{n , Ω}^{′} / {\tilde{P}}_{n , Ω} ) ] .\end{matrix} \qquad Equation 6$$

Here, Ũ′ is the modified control signal with necessary corrections. This mapping is conducted for all external conditions and the corresponding correction factors are tabulated. Similarly, the frequency response is measured under different conditions. This provides a complete mapping of the control signal corresponding to the basic raw engine sound.

Sound may controlled to either attenuate or enhance the sound at any given speed and order. Both of these functions may be represented by a single gain parameter β (block 20), where

β=β(ωn) Equation 7.

A value of β=1 implies no control (unity gain), β<1 implies attenuation and β>1 implies sound enhancement. Thus, β=0 will be equivalent to full cancellation. The net control signal is derived from the baseline value and the β value by:

\tilde{V}′_{n,ω}=(β_{n,ω}−1)×Ũ′_{n,ω} Equation 8.

Here, Ũ′_{n,ω} includes the effects of all external parameters, and \tilde{V}′_{n,ω} is the final control signal. Clearly, for β<1, Equation 8 will yield a negative control signal, which has the same effect as an out-of-phase sound wave. Once the vehicle parameters have been determined, the only user input is the β table for obtaining the desired sound characteristic. The system 10 also includes an audio amplifier 24 that covers the entire frequency range of interest so that a sufficient audio output is provided by the speaker 22.

As such, the engine sound, frequency response and vehicle effects are each decomposed into engine orders (multiples of engine fundamental frequency) and control is executed for each individual order independently.

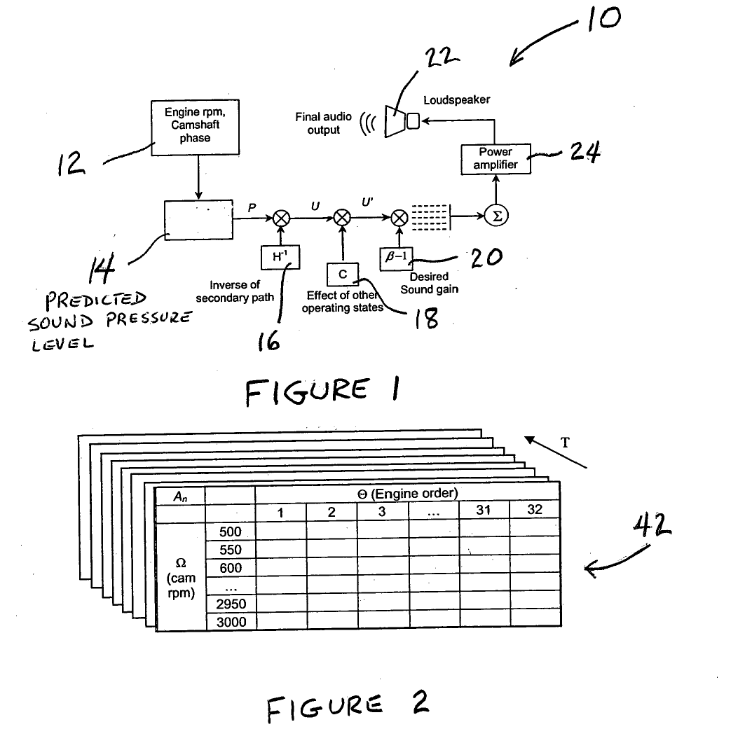

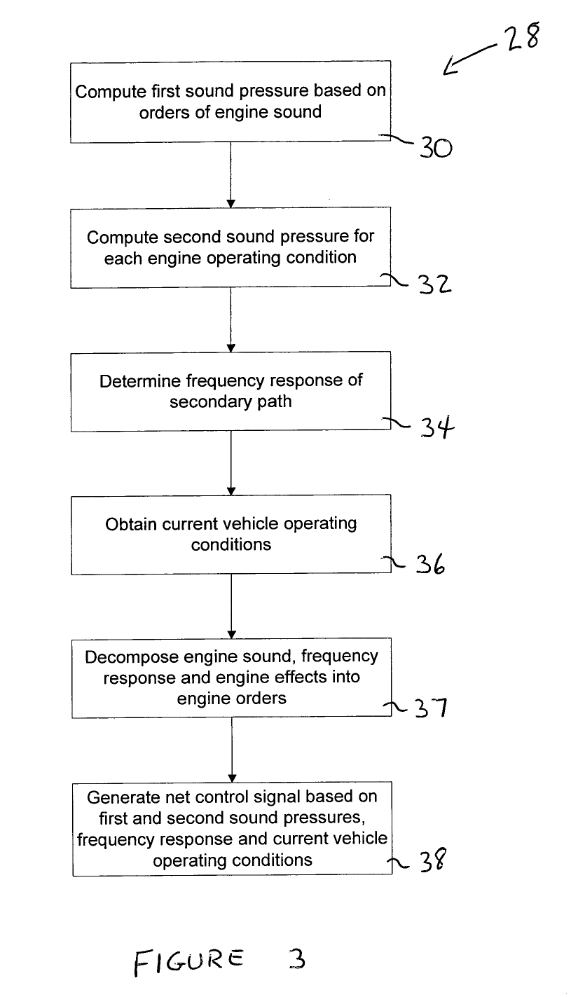

In accordance with the invention, an algorithm is implemented in real-time at a sampling frequency that is sufficient according to the Nyquist criterion. Other algorithm refinements include data interpolation for different vehicle parameters, accurate rpm and phase determination from the cam signal and incorporating time delays between different engine events. The system 10 may be controlled by a conventional automotive microcontroller having sufficient internal memory to hold all data tables. Referring to FIG. 2, an example of a set of look-up tables 42 for temperature effects on control signal amplitude is shown. A transceiver is used for transmitting and receiving signals from the vehicle databus. Finally, a digital anti-imaging filter is used at the output with an upsampling filter, so that a simple low-order analog filter can be used for the analog signal.

Referring to FIG. 3, a flow diagram 28 depicting aspects of the present invention is shown. In step 30, a first sound pressure is computed based on orders of sound generated during a run up of the engine. The engine is then operated under a plurality of operating conditions and a sound pressure is computed for each of the operating conditions (step 32). A frequency response of the secondary path previously described in relation to FIG. 1 is then computed (step 34). Next, current vehicle operating conditions are obtained from the vehicle databus at step 36. The engine sound, frequency response and vehicle effects are each decomposed into engine orders (multiples of engine fundamental frequency) at step 37. At step 38, a net control signal is computed in real time based on each of the first and second sound pressures, the frequency response and the current vehicle operating conditions to either attenuate sound or enhance sound, wherein each individual order is controlled independently.

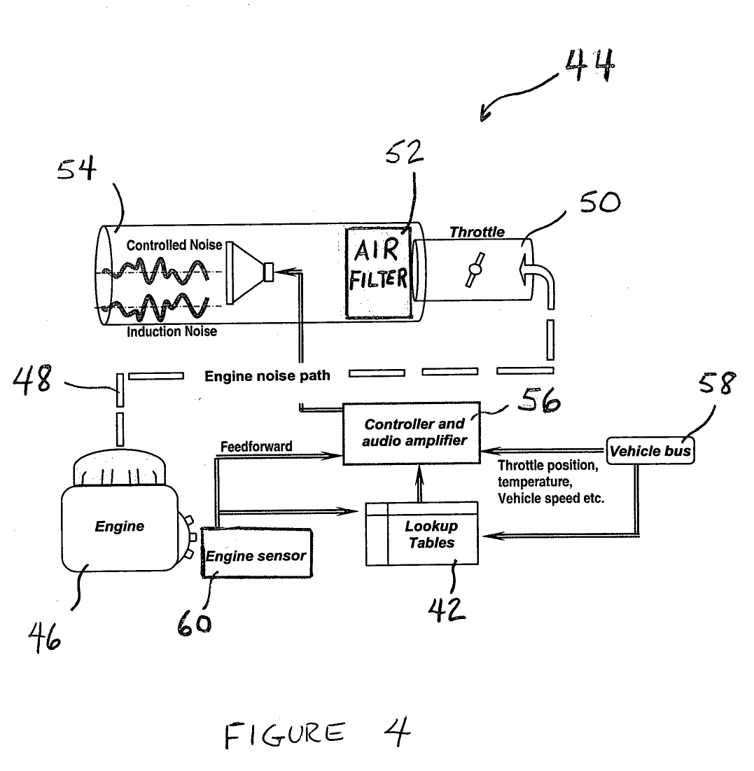

Referring to FIG. 4, a schematic representation for a feedforward active noise control system 44 is shown. Induction noise is generated by the reciprocating motion of intake valves of an engine 46. The induction noise travels along an engine noise path 48 and through a throttle 50 and air filter 52 of an air induction system 54. The speaker 22 is located in the air induction system 54 and serves to generate an audio output adapted to either attenuate or enhance the induction noise. In accordance with the current invention, sound pressure is computed for each order of induction noise of the engine 46 during a gradual run-up of the engine 46. The data is arranged as amplitude and phase with respect to a reference signal provided by an engine sensor 60. The reference signal is indicative of an angular position of a camshaft that drives the intake valves of the engine 46. The system 44 also includes a controller and audio amplifier module 56 having a controller which controls the system 44. The controller includes sufficient internal memory to store the data tables 42 generated in accordance with the current invention. Current vehicle operating conditions are obtained by a transceiver from a vehicle databus 58 and are provided to the controller. Induction noise, frequency response and vehicle effects are each decomposed into engine orders and a net control signal is then computed as previously described. The net control signal is amplified by an amplifier in module 56 prior to being provided to the speaker 22. The speaker 22 then generates an audio output which is adapted to either attenuate or enhance induction noise.

It has been established that with the knowledge of all necessary operating conditions, variations in vehicle intake sound are minimal over time and amongst actual vehicles. There is likely to be some statistical variation amongst vehicles which will persist as an error in the system. It has been determined that the effect of such residual errors is acceptable in view of other performance benefits of the system.

In this manner, a control signal having a desired profile may be provided for sound corresponding to each of the engine orders and half-orders, which are known to dominate the induction sound spectrum. This results in a reduction in vehicle sound emission, elimination or simplification of passive system parts and gives the vehicle a desired specific acoustic character.

The current invention is readily adaptable to different types of vehicles, including passenger sedans and sports utility vehicles, as well as different engine types. In addition, the current invention provides a robust low-cost solution for controlling engine intake sound.

Thus it is apparent that in accordance with the present invention, an apparatus that fully satisfies the objectives, aims and advantages is set forth above. While the invention has been described in conjunction with specific embodiments, it is evident that many alternatives, modifications, permutations and variations will become apparent to those skilled in the art in light of the foregoing description. Accordingly, it is intended that the present invention embrace all such alternatives, modifications and variations are far within the scope of the appended claims.

What is claimed is:

1. An active noise control system for controlling induction noise of an internal combustion engine, comprising:
a speaker; and
a controller for generating a control signal that drives said speaker, wherein said signal is based on at least one current vehicle operating condition, a determination of a first sound pressure for each order of sound generated by said engine during a run up of said engine and a determination of a second sound pressure computed for each of a plurality of operating conditions of said engine, wherein said signal controls each of order of sound generated by said engine independently to drive said speaker to generate an audio output to control said noise.

2. The system according to claim 1, wherein said signal is also based on a frequency response of a microphone and a speaker used in computing said first and second sound pressures.

3. The system according to claim 1, wherein said vehicle operating conditions are obtained by a transceiver from a vehicle databus.

4. The system according to claim 1, wherein said signal includes a gain factor for attenuating said sound.

5. The system according to claim 1, wherein said signal includes applying a gain factor for enhancing said sound.

6. The system according to claim 1, wherein said signal includes a correction factor for each of said operating conditions.

7. The system according to claim 1, further including an amplifier for amplifying said signal.

8. The system according to claim 1 wherein said controller decomposes said first and second sound pressures and generates look-up tables.

9. The system according to claim 1 wherein said controller utilizes an algorithm that uses a Nyquist criterion.

10. The system according to claim 1 further including a time delay between said engine operating conditions.

11. An active noise control system for controlling induction noise of an internal combustion engine, comprising:
a speaker; and
a controller for generating a control signal that drives said speaker, wherein said signal is based on at least one current vehicle operating condition, a determination of a first sound pressure for each order of sound generated by said engine during a run up of said engine, a determination of a second sound pressure computed for each of a plurality of operating conditions of said engine, and a determination of a frequency response of a microphone and speaker used in determining said first and second sound pressures, wherein said signal controls each of order of sound generated by said engine independently to drive said speaker to generate an audio output to control said noise.

12. The system according to claim 11, wherein said vehicle operating conditions are obtained by a transceiver from a vehicle databus.

13. The system according to claim 11, wherein said signal includes a gain factor for attenuating said sound.

14. The system according to claim 11, wherein said signal includes applying a gain factor for enhancing said sound.

15. The system according to claim 11, wherein said signal includes a correction factor for each of said operating conditions.

16. The system according to claim 11, further including an amplifier for amplifying said signal.

17. The system according to claim 11, wherein said controller decomposes said first and second sound pressures and generates look-up tables.

18. The system according to claim 11, wherein said controller utilizes an algorithm that uses a Nyquist criterion.

19. The system according to claim 11, further including a time delay between said engine operating conditions.

20. An active noise control system for controlling induction noise of an internal combustion engine, comprising:
a speaker located within an air induction system;
a controller for generating a control signal that drives said speaker, wherein said signal is based on at least one current vehicle operating condition, a determination of a first sound pressure for each order of sound generated by said engine during a run up of said engine, a determination of a second sound pressure computed for each of a plurality of operating conditions of said engine, and a determination of a frequency response of a microphone and speaker used in determining said first and second sound pressures, wherein said signal controls each of order of sound generated by said engine independently to drive said speaker to generate an audio output to control said noise;
a sensor for providing a reference signal indicative of a camshaft position, wherein said reference signal is utilized in determining said first pressure; and
a transceiver for providing said at least one current vehicle operating condition to said controller.
